(19) United States
(12) Patent Application Publication
(10) Pub. No.: US 2001/0049096 A1
(43) Pub. Date: Dec. 6, 2001

(54) SAMPLE IDENTIFICATION WITH ANALYTE DETERMINATION

(76) Inventor: STEPHEN J. BROWN, WOODSIDE, CA (US)

Correspondence Address: BLACK LOWE & GRAHAM, 816 SECOND AVE., SEATTLE, WA, 98104, US

(21) Appl. No.: 09/274,869

(22) Filed: Mar. 23, 1999

(51) Int. Cl. C12Q 1/68

(52) U.S. Cl. 435/6

(57) ABSTRACT

A system is provided for sample identification and at least one other piece of information, e.g. analyte determination, relevant to a source of a sample, which information is posted to a database. The database can compare the sample identification information with a prior control to see whether the alleged source of the information is correct. The sample identification and the analyte determination are made with the same sample. The identification is achieved by analyzing for invariant markers, such as public antigens and DNA, which have sufficient variety in the population, so as to provide reasonable assurance of the source of the sample. At the same time an analyte may be determined, so that the information is coordinately received. A simple self-contained device may be employed where the sample is divided into two portions and each analyzed separately on separate tracks. For identification, the distribution of bands or spots having the labeled reagent can serve to identify the source of the sample. Conventional assays on solid supports can be used for identifying the analyte. Using simple instrumentation, the tracks can be read and the information transmitted to a central database.

### TECHNICAL FIELD

The field of this invention is the correlation of sample identification and analyte determination.

### BACKGROUND

The diagnostic industry has greatly expanded its ability to identify analytes in numerous contexts and media using an expanding variety of chemistries and protocols. One of the needs is the ability to identify a sample with the individual source of the sample. This problem requires coding and identification being attached to the sample and the processing and result from the assay of the sample. While this is possible in a structured environment, such as a clinical laboratory or a hospital, even in these situations where great care is taken to maintain continuity from the time of taking the sample to the time of obtaining a result, errors can occur. The problem is increased where the same sample is to be used in a plurality of determinations, which may require separate handling and processing.

There is the further situation where the source of the sample may have an interest in providing an erroneous result. For example, for many jail inmates and parolees, there is an interest in monitoring the use of illicit drugs. To require that the individual appear at a site and urinate under supervision is undesirable. If one could be certain that the urine sample had not been tampered with, this embarrassing situation could be avoided. There are other situations where large numbers of samples are received within a short time interval, where it would be desirable to be able to independently identify the sample source, where there was any ambiguity as to the source.

Also, one may wish to be able to make the determination at a site distant from the site where the information will be analyzed. In this situation one would like an automatic device which would perform the analysis and a simple reader which could read the result and transmit the result by communication lines to the information, receiving site.

Depending on the nature of the device, it could be placed in the individual's home or at a central site, such as a police station, library, or other relatively ubiquitous public site. There is, therefore, an interest in developing methodologies which provide independent identification of a sample as to its source, which is reliable, cannot easily be faked, and preferably can be used with simple instrumentation.

### PRIOR ART

U.S. Pat. No. 5,270,167 describes patient identification by detecting an array of antibodies present in the patient's urine. Articles describing HLA and their detection in urine include Park, et al., Nephron 1998; 79:44-9; Tsongalis et al., J Forensic Sci 1996 avazava et al., J Clin Lab Anal 1994; 8:432-6; Dautigny et al., Biomedicine 1979; 31:233-6; Vincent and Revillard, Transplant Proc 1979; 11:1301-2; Lamm and Kristensen, HLA system—new aspects 1977 Jul 29, Formal Genetics of the HLA System. pp. 1-20; and Reisfeld, et al., J Immunol 1977; 118:264-9.

Devices that may be used for detection of proteins include U.S. Pat. Nos. 5,096,833; 5,132,086; 5,215,102; 5,409,664; 5,416,000; and 5,593,895.

### SUMMARY OF THE INVENTION

Methods and devices are provided for identification of a mammalian source and, optionally, an analyte from a physiological sample. In addition, one can determine location and time for the sample determination. The methods employ identification of public markers, as exemplified by the use of monoclonal antibodies for the identification of proteins in a physiological sample, where the proteins are classified as being members of a class of proteins for which there is sufficient variety in the population for identification and concurrent analyte detection, usually as an immunoassay. Alternatively, one may use DNA detection, employing microfluidic devices having sample preparation. By detecting the public markers and/or DNA and a ligand of interest together, one can be assured of the source of the sample. This is exemplified by providing a bibulous pathway, where binding of the proteins at specific sites provides an identity pattern for the source, while the immunoassay provides for identification of the analyte. The result may be read by a reader with simple signal detection and processing, desirably being able to transmit the information to a central source.

### BRIEF DESCRIPTION OF THE FIGURES

FIG. 1 is a flow chart demonstrating the method of this invention;

FIG. 2 is a diagrammatic plan view of a bibulous strip according to this invention; and

FIG. 3 is a plan view of a portable device for assaying for the protein markers and the analytes.

### DESCRIPTION OF THE SPECIFIC EMBODIMENTS

Systems are provided for the concurrent determination of identification of the source of a physiological sample and, optionally, the presence of one or more analytes, the location of the determination, and or the time. A physiological sample is analyzed for markers identifying the human source of the sample, by employing markers which are distinctive for an individual in a group of interest, which can be determined with devices which are easy to employ and provide a detectable signal associated with a pattern of markers defining the source of the sample. The markers may be antigens or DNA, where there is sufficient diversity to allow for reasonable identification and certainty as to the source in the group of interest. The group of interest will be at least about 100 people and may be 1,000 or more. Detection will involve a plurality of identifying markers which provide a pattern of binding distinctive for the individuals in the group, as well as others in the population at large. The information concerning the source of the sample may be combined with one or more other pieces of information, such as the presence or absence of one or more analytes of interest, the location and/or time of making the determination, or other information which may be relevant to the source of the sample.

The system employs means for detecting ubiquitous markers, which are invariant over time, such as public antigens and DNA, while at the same time, determining one or more analytes of interest from the same sample. Conveniently, a detection device which has members of a specific binding pair, ligand and receptor, where ligand is the host marker which identifies the source or the analyte of interest, and receptor may be any molecule which has a high affinity for a particular conformation and charge and polarity distribution, usually an antibody, although other proteins may find use, such as enzymes and surface membrane receptors. The detection device will identify proteins, which are specific for the individual, which proteins will occur in sufficient variety to give a reasonable expectation of uniqueness. In addition, the device will identify analytes of interest, so that one can relate the presence or absence of the analyte to a particular individual source. The detection of the ligands and the receptors will depend upon cooperation between the ligand of interest and its receptor(s) or a competition between the ligand of interest and a competitive binding moiety for a common receptor, where one of the members of the specific binding pair is labeled with a detectable label. In many situations, the device will be capable of being read by a signal detection device, which may also serve to communicate the results to a remote database.

The identification of a physiological sample source is provided by identifying proteins in the sample, where the proteins serve as protein markers for the individual source of the physiological sample. For the most part, the proteins will be “public” proteins, rather than private proteins. That is, the proteins are characterized by being members of a class having a plurality of members, members of the class being present in all subjects and having a reasonable variety to allow for identification without an undue number of determinations. In addition to the identification of the source, an analyte is also determined which is associated with the sample source. The proteins are identified with monoclonal antibodies or competitive binding moieties, which are patterned on a bibulous support, where the pattern of binding of the protein markers in the sample identifies the source. The competitive binding moieties are molecules, which share at least one epitope with the protein markers. The analyte will usually be determined by a convenient immunoassay, where both determinations may be made in a single pathway or two separate pathways.

There are numerous families of proteins associated with mammals, such as the major histocompatibility (MHC) proteins (for humans referred to human lymphocytic antigens [HLA]), the immunoglobulin superfamily, particularly the allotypes, blood types, allelic surface membrane proteins, etc. Of particular interest are the HLA proteins, which may serve as paradigmatic of other groups of proteins which fulfill the characteristics of this invention. Desirably the proteins of interest have few or a single common epitope, so that a single binding protein, e.g. a monoclonal antibody, will bind to all of the members. The Class I HLA have β₂-microglobulin common to the Class I HLA antigens. The Class I HLA antigens are divided into groups A, B and C, and each group has numerous polymorphic proteins as an α-chain. These are public antigens in that each person has six Class I HLA antigens, two in each group, where the variety of combinations offers assurance of a unique combination, except for identical twins. If twins were a problem for distinguishing, one could distinguish them by one or more antibodies that were prevalent in one twin and not in the other. However, since twins are only a minor portion of the population and usually will not be involved at the same time where identification is required, for the most part use of the HLA antigens will be sufficient.

There are monoclonal antibodies for most of the known HLA antigens and in most cases, the monoclonal antibodies are specific for a particular polymorphism. In those instances where the only available monoclonal antibodies cross-react with two HLA polymorphs, it will be understood that the antibody cannot differentiate between the two polymorphs and there will be ambiguity as to these polymorphs. However, since it is believed that in most instances it will be sufficient to have 2 or more markers, preferably at least 3 markers, to identify an individual, ambiguities as to particular polymorphs can be tolerated. With larger groups one may need to identify from 4 to 76 markers. The largest variation is Class IA, with lesser variation with Classes IB and IC. Depending on the size and source of the population it may be sufficient to have antibodies for IB and IC or only for IA. By using only monoclonal antibodies to the IA HLA antigens, one may be able to distinguish relatively large groups with reasonable certainty of identification of the individual. The uniqueness would be greatly expanded by having antibodies to the most prevalent IB and/or IC antigens.

To identify the individual, one could provide spaced-apart bands or spots of monoclonal antibodies on a bibulous surface. For each band or spot, one would use a monoclonal antibody composition specific for a particular HLA antigen. The bibulous support would be exposed to the sample, whereby HLA antigens present in the urine would bind to their complementary antibodies. After washing away any non-specifically bound protein, one would add labeled anti (β₂-microglobulin), which would bind to any HLA antigens present on the support. By detecting the label and knowing the spatial arrangement of the monoclonal antibodies, a particular pattern of the label sites, bands or spots, would be indicative of the individual. With bands one would have a bar code, while with spots, there would be a spatial orientation.

Alternatively, one could have competitive binding members bound to the bibulous support, which compete for antibodies to the different α-chains of the HLA antigens. The reagent would be labeled antibodies, so that in the presence of the specific HLA antigen, the binding sites would be filled and the antibody conjugate would not bind to the competitive binding member on the support. In this instance, the absence of a signal would be indicative of the presence of the HLA antigen.

Depending on where the sample is to be handled and the sophistication of the person doing the processing, whether a technician in a laboratory or hospital, the individual from whom the sample is taken, or a trained lay person, and the equipment available to the person, the sample may be treated in a variety of ways. For example, a urine sample may be filtered, concentrated, diluted with buffer, and the like. A blood sample may be filtered to remove red blood cells, stabilized with citrate, etc. For the most part, urine samples will be used. One may employ manual washing steps, addition of reagent, or the like.

In order to ensure that the urine sample is a fresh sample, a pH detector may be employed, which is conveniently a dye, which is pH sensitive. Desirably, the dye is fluorescent and will fluoresce at a different wavelength, depending on the pH of the urine. Since urine which has been allowed to stand for some time increases in basicity, one need only have a dye which changes its emission wavelength at basic pH (≧7). For dyes, the absorption wavelength would have to change at basic pH. Therefore, on the bibulous support would be a dye, which would respond to the pH and turn color if the basicity was significantly above normal.

Various devices may find use, depending upon the purpose for the determination. Where the information is for the use of the assay performer, then the device will depend on the equipment available for the determination and the sophistication of the assay performer. Where the device is to be used by the source of the sample, ordinarily the device will be self-contained, usually permitting instrumented reading of the device, so that the results may be recorded, and in many instances, transmitted to a remote database.

A device, which may be employed for the purposes of the subject invention, would have a sample receiving port, which would remove red blood cells from a blood sample and might filter urine to ensure the absence of particulate matter. The sample would wet the bibulous support and buffer and reagent would then flow through the sample site, carrying the components of the sample into the detection area. As described above, the detection area could have bands or dots of different antibodies, each antibody specific for one or a few HLA antigens. The reagent in the buffer solution would bind to the HLA antigens present in the sample, so that the complex of the HLA antigens and the anti (β₂-microglobulin) would bind to the specific monoclonal antibodies in the detection area. By having the anti (β₂-microglobulin) conjugated to fluorescers, fluorescent particles, opaque particles, colored particles or other label which provides for ready detection and a high gain between the number of HLA antigens and the number of labels, the individual bands or spots can be readily detected. The desired gain will be modified in accordance with the degree of background that can be tolerated, while still observing the signal.

The background will be affected by the non-specific binding of the label conjugate to the bibulous support, the use, volume and efficiency of a wash medium to carry the label conjugate, the intensity of the signal at the band or spot, and the like. Desirably, one would follow the reagent solution with a buffer wash solution to reduce the amount of labeled conjugate, which is non-specifically bound in the detection area. Alternatively, one could modify the area between bands and spots so as to reduce the background. The modification will depend on the nature of the label. For example, if the label is a fluorescer, one might add quencher to the intervening regions. If the label is a dye particle, then the intervening regions would have a dye which would give a different signal, so that when the detection area is scanned the regions which had the additional dye would be ignored. However, since these modifications add cost to the device, using an effective wash solution will be preferred.

Instead of a bibulous support, one or two capillaries maybe used. The reagents may be banded on the surface of the capillary, by treating the capillary walls with a light-activated linker, which forms a reactive intermediate when irradiated. Various diazo or triazo compounds may be used to react with the reagents, particularly proteins. One immerses one or a group of capillaries in a reagent solution and allows the reagent solution to rise to a particular height or the length of the capillary. By irradiating at a particular height, the reagent in the solution will become bound at that position, forming a band of reagent around the capillary. One withdraws the reagent solution from the capillaries and repeats the process for a different reagent solution, until all of the necessary reagent bands have been created. One may have one capillary for all of the reagents or two capillaries, one for the analyte(s) and one for the sample identification. Instead of coating the walls of the capillaries, one may fill the capillaries stepwise with a powder impregnated with reagent, providing bands of powder with different reagents at different heights in the capillary. The powder would be absorbent, so that the buffer eluent and wash solutions would be absorbed through the capillary column.

The sample can be divided into two pathways. One pathway is the sample identification pathway and the other pathway would be the analyte identification pathway. The analyte identification pathway would be arranged in the same manner and would be subject to the same reagent solution. However, the reagent relevant to this region could be a variety of different reagents and different protocols cold be used. Since a number of analytes of interest are haptenic, that is small organic molecules of less than about 2 kD, one cannot bind at two different epitopic sites, as with antigens. Therefore, one can have antibody to the analyte, so that analyte, which is present, binds to the antibody and prevents the antibody in the reagent solution to bind to analyte present in the detection system. Alternatively, and less desirable is to have the label bound to a labeled competitive binding moiety and antianalyte present in the detection region, so that the absence or diminution of signal at a band or spot is indicative of the presence of the analyte. Either protocol will work, but the use of labeled antianalyte and analyte in the detection are preferred.

The analyte may be haptenic or antigenic, depending on the analytes of interest. One group of analytes of interest are drugs of abuse, which may include drugs which are given under prescription. These drugs include cocaine, heroin, amphetamine, methamphetamine, LSD, NMDA, phencyclidine, barbiturates, benzdiazepines, carbmazepines, tricyclic antidepressants, or any other drug on the interdicted listed or which may be abused. Other drugs of interest may be therapeutic drugs, where a patient is required to take a plurality of drugs and the patient needs to be monitored. There may also be interest in blood antigens, which may be associated with neoplasia, hyperplasia, cardiological conditions, neurological conditions, metastatic conditions, alveolar conditions, immunological conditions, etc. In each case the analyte or antianalyte would be placed at a specific site in the detection region, so that the presence or absence of a signal could identify the presence of the particular analyte.

It should be understood that the invention is being described in relation to a specific device, which provides for ease of use and the ability to be performed by a lay person, as well as being read in a convenient manner. Where a sophisticated person or laboratory is involved, some of the steps may be performed manually, such as washing and additional washings employed. The bibulous support may be dipped into a solution, a wash buffer moved through the bibulous support using a centrifuge or other device for moving the fluid through the bibulous support, or other procedure, as appropriate. Furthermore, the system need not require a device which acts substantially independent of the user, but may depend on the user to dispense sample, reagent, perform individual acts or make observations, or the like.

Instead of using proteinaceous public antigens, one may use DNA. DNA may come from any convenient source, physiological fluids, such as blood, saliva, etc., solid cellular samples, such as skin scrapings, hair, etc. There are a large number of systems for sample preparation of DNA and identifying sequences present in DNA. Simple devices are employed for denaturing the DNA and performing amplification of portions of the DNA, using primers, where the primers may be labeled for detection. Methods for preparing and detecting DNA samples may be found in U.S. Pat. Nos. 5,202,231; 5,576,197; 5,631,134; 5,837,832; 5,871,928; 5,872,010; and 5,876,930. The use of microfluidic devices for analysis is described in U.S. Pat. Nos. 5,842,787; 5,871,697 and 5,842,787.

Where sufficient DNA is available, chromosomes may be fragmented by mechanical shearing and the resulting DNA fragments denatured and combined with a DNA array, which allows for distinguishing the group of interest from each other, as well as the public at large, with a reasonable degree of confidence. One can have complementary DNA sequences hybridized to the DNA array, where a quencher is provided on the complementary DNA and a fluorescer on the DNA oligonucleotides bound to the support. By denaturing the array in the presence of the sample DNA, those areas which become fluorescent indicate that there is complementary DNA in the DNA sample. One can then obtain a pattern of fluorescence associated with an individual, which is substantially distinctive for the individual. By having a simple fluorimeter, e.g. a CCD camera with a laser source, the pattern may be detected and the information sent to a database for analysis.

One may combine the detection device with a device which permits identification of the location at which the determination has been made and/or the time the determination has been made. By using a device which employs the global position system (GPS), one can transmit the information concerning the source, as well as information as to location and/or time by transmitting the information received from a satellite source of the location and/or time.

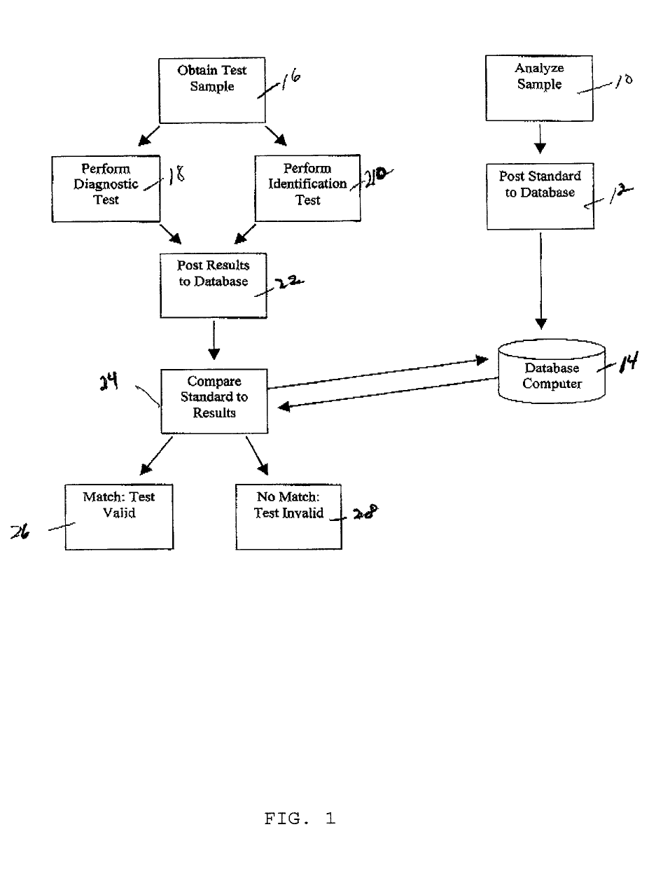

A system employed for identifying and analyzing physiological samples is set forth in FIG. 1.

In the flow chart, FIG. 1, one obtains an initial physiological sample 10 from the subject to compare future samples for identification. The results of the test are posted 12 to the database computer 14. Subsequently, a new sample is obtained 16 and the sample concurrently subjected to a diagnostic analysis 18 and an identification test 20, which is substantially the same identification protocol as was used with the initial sample 10. The results are posted to the database 22 followed by comparing 24 the results of the subsequent sample with the results stored in the database 14. If there is a match 26, the results of the diagnostic test are treated as valid for the subject. If there is no match 28, the results are treated as not relevant to the subject.

For further understanding of the invention the drawings of exemplary devices will now be considered. The drawings are in part taken from U.S. Pat. Nos. 5,132,086 and 5,409,664, which are specifically incorporated by reference in their entirety, and modified to be applicable to this invention.

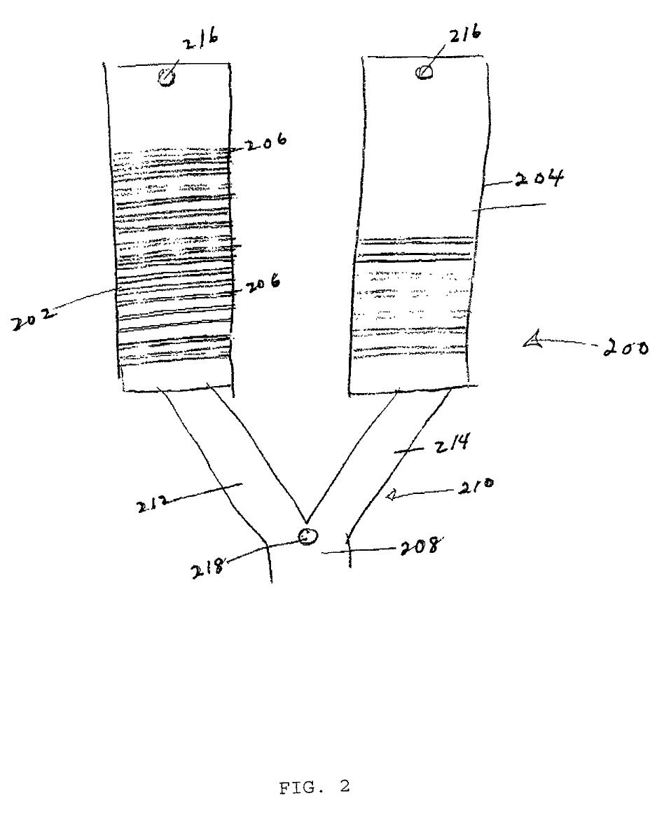

FIG. 2 is a diagrammatic plan view of a bibulous strip according to this invention. The strip 200 has a subject identification track 202 and a analyte analysis track 204. The tracks are made of bibulous material which will move aqueous solutions and their contents by capillary action. Once wetted, the strip 200 will move the aqueous solution along the tracks 202 and 204 until the solution is exhausted. The identification track 202 has a plurality of bands 206. Each band substantially completely crosses the identification track 204, so as to require any of the aqueous solution to encounter the composition of the band. Each band will be at least about 0.1 mm wide, frequently at least 0.25 mm wide, in the direction of flow, and will usually not exceed 2 mm, more usually not exceed 1 mm, generally ranging from about 0.25 mm to about 0.75 mm, depending on the amount of the reagent placed at the site, the expansion upon absorption of the reagent solution by the bibulous material, the number of bands necessary to be placed on the strip, the distance that the components of the solution can effectively travel and the like. Desirably, the band should be as thin as detection of the band permits and can be effectively laid down on the bibulous material.

The bands may be applied by spraying e.g. ink jet spraying, silk screening, or other conventional technique. Each band is separately applied to its site, there being sufficient separation between bands to prevent mixing of the different reagent solutions. The bands may be applied simultaneously or consecutively, conveniently using a solvent which rapidly dries or maintaining the substrate at a moderately elevated temperature to enhance evaporation of the solvent. The faster the band dries, the less widening of the band that will occur and the less likelihood for mixing of different reagent solutions. Desirably, each band will be separated by at least 0.25 mm and not more than 2 mm, usually not more than 1 mm, the spacing being consistent with detection of the signal and absence of mixing of the solutions. Depending on the identifying components of the sample, there will be at least 10 bands, usually at least 20 bands and there may be 50 bands or more, preferably not more than about 30 bands. Generally, each band will have at least about 10¹⁰ molecules, preferably at least about 10¹² molecules and may have 10¹⁶ molecules, or more. The solutions, which are applied, will usually be at least about 0.01 μM, generally at least about 0.01 mM and not more than about 1 M.

Other agents may be present in the reagent solution to fulfill a variety of purposes. Included with the reagents may be detergents to provide better absorption, where the detergents may be non-ionic, cationic or anionic, innocuous proteins to provide better availability of the reagent in the band and ease of handling, stabilizers, preservatives, such as sucrose, trehalose, etc., and buffers, e.g. phosphate, tris, etc. These components are conventional and will generally vary in concentration from about 1 μM to 100 mM.

The band compositions will be selected so as to have greater spacing between the more prevalent members of the identifying group of reagents and desirably will have the members of the group, which are less prevalent and therefore more revealing as to identity, closer to the source of sample. The sample is received at the base strip 208 of the yoke 210, having a first arm 212 going to the identification track 202 and a second arm 214 going to the analyte analysis track 204. The yoke 210 is of bibulous material so that the sample and the eluent/reagent solution which will be picked up by the base strip 208 will travel across the yoke 210, being distributed into the two arms 212 and 214 and then into the two tracks 202 and 204. The eluent including the reagents, which follows the sample, will carry the components of the sample with it and be of sufficient volume to carry the sample past all of the bands in both tracks and may extend to at least approximately the end of the tracks. At the end of each track, a spot 216 is provided, so that upon fluid reaching the spot 216, the spot changes its color to indicate that the determination is completed. Alternatively, when a wash solution is used, one may wish to have the eluent/reagent solution be exhausted prior to the spot indicating completion, so that the liquid front of the wash solution is necessary for indicating the completion of the assay. In addition, one may have a spot to detect whether a urine sample is fresh. This urine detection spot could be anywhere, but is conveniently at the top of the base at site 218. If the spot indicates that the sample is not fresh, the assay is aborted and no readings are taken. For urine various pH sensitive dyes can be used to detect that the sample has been stored for some period of time.

The analyte analysis track 204, also has a series of bands for each of the analytes of interest. The reagent is selected to specifically react with the analyte or its specific binding member. For example, where one is interested in drugs of abuse, one could have labeled anti (drug) as the reagent, which in the presence of the drug in the sample would react and the binding sites would be filled. The bands would be the different drugs. Unfilled binding sites of the reagent would then be captured by the drug in the band, creating a detectable band in the presence of drug, but the absence of a detectable band in the presence of drug. Alternatively, one could have anti (drug) present as the band and a conjugate of the drug with a label as the reagent. The drug and conjugate would compete for a limited number of binding sites, so that a reduction in signal would indicate the presence of the drug. Alternative protocols are also available.

Since there will probably be fewer analytes to be detected, the bands need not be as thin as the bands for identification, and need not be spaced as closely. However, there is no need to use excessive reagent, so the bands may be the same size and spaced apart about the same as the identification bands, usually being less than about twice the width and spacing of the identification bands.

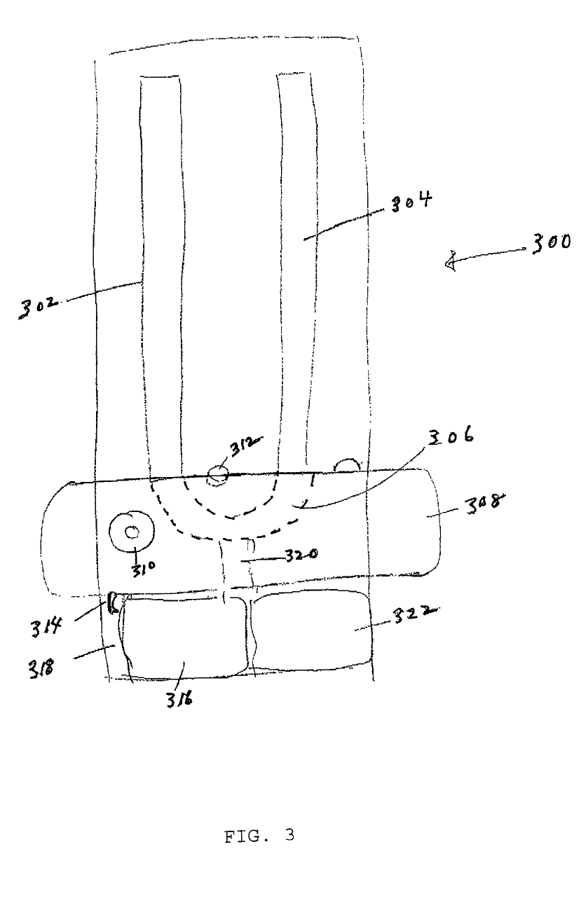

FIG. 3 is a plan view of a portable device, which may be used with a spectrophotometer to analyze the sample. The device 300 has tracks 302 and 304, comparable to the tracks 202 and 204 shown in FIG. 2. The yolk 306, comparable to yolk 210, is shown in broken line hidden by the slide. The slide has a sample receiving port 310 and a spring loaded ball 312. The sample port may have some glass wool to filter out red blood cells, in the event that whole blood is the sample. The slide also has razor 314. When the slide 308 is moved from left to right, the razor first cuts reagent solution bag 316, which sits in well 318. In performing the assay, one moves the slide 308 so that the sample port is over the base 320 and adds the sample. At the same time the reagent solution bag 316 is cut and the solution is released into the well 318. The base 320 extends into the well and absorbs the solution. The solution moves through the base and the sample, carrying the sample through the yoke 306 into the tracks 302 and 304. The solution will contain whatever reagents are necessary for the determination of the identification of the sample and the analyte(s) present in the sample. The amount of liquid in the reagent solution will be sufficient to carry the sample past the bands, but not to the end of the tracks 302 and 304. After the reagent solution is substantially exhausted, which will be indicated by the liquid front in the tracks, the slide may be moved further, cutting the second bag 322 containing the wash solution. The base 320 will now absorb the wash solution which will be transported to the end of the tracks 302 and 304, indicating that the assay is complete. The wash solution will serve to remove non-specifically bound labeled components from the bands and spaces between the bands. In this way, the device is ready to be read in an appropriate detection instrument.

For reagents which are labile in water, one may not wish to have a reagent solution containing such labile reagents. In this situation, one may introduce the reagents in the well, as a formulation which will rapidly dissolve in the eluent solution. Some of the reagents may be in solution and the labile reagents provided as a powder in the well, where the formulation of the powder allows for rapid dissolution of the reagents into the eluent. This can be achieved by having frothing compositions, such as carbonate and acid, mixtures with water miscible materials, such as sugars, etc. Alternatively, one may use a non-aqueous water soluble solvent to form a solution of the reagent, so that the eluent will readily dissolve the reagent upon release into the well.

Where the detection instrument has the capability of analyzing the bands and transmitting the information to a central data processing unit, the identification information and analyte information will be substantially simultaneously received and recorded together, conveniently as a single record. In this way, individuals to be assayed, distant from the information receiver may be monitored without requiring the individual's attending a particular site or having to travel to a distant site for analysis.

By using the subject device or other devices having comparable capabilities, sample identification can be routinely and credibly determined in conjunction with the determination of an analyte. By having the identification of the sample coupled with the assay of the analyte in a single instrument, there is very little probability of misidentification or the results becoming separated.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A system for combining identification of the source of a sample and one other piece of information relevant to the donor, employing a device which is capable of detecting in a physiological sample biological information which distinguishes the source of the sample from at least a group of interest, said system comprising:
performing an analysis of said sample to identify the source of said sample, whereby data is obtained relevant to said source;
posting said data and at least one other piece of information relevant to said source to a database;
comparing said data to a prior standard related to said source; and
determining whether the data is from the source of said standard.

2. A system according to claim 1, wherein said analysis concurrently determines the presence of an analyte of interest.

3. A system according to claim 1, wherein said at least one other piece of information is the location or time at which said performing occurred.

4. A system according to claim 1, wherein said analysis is the analysis of public antigens.

5. A system according to claim 1, wherein said analysis is the analysis of DNA.

6. A system according to claim 1, wherein said analysis concurrently determines said source of said sample and an analyte in said sample.

7. A system for combining identification of the source of a sample and the presence of at least one analyte in said sample, employing a device which is capable of detecting in a physiological sample biological information which distinguishes the source of the sample from at least a group of interest and concurrently said at least one analyte, said system comprising:
performing an analysis of said sample to identify the source of said sample, by dividing said sample into two parts and detecting the binding of public antigens using antibodies and said at least one analyte using antibodies, whereby data is obtained relevant to said source and said at least one analyte;
posting said data related to said source and said at least one analyte;
comparing said data related to said source o a prior standard related to said source; and
determining whether the data is from the source of said standard, where if the data is from the same source the data is accepted.

8. A system according to claim 7, wherein said public antigens are HLA antigens.

9. A system according to claim 8, wherein said at least one analyte is a drug of abuse.

10. A system according to claim 8, wherein said performing is with a bibulous strip to which antibodies are bound which bind to at least 20 different HLA antigens.
